(19) United States
(12) Patent Application Publication
(10) Pub. No.: US 2024/0368665 A1
(43) Pub. Date: Nov. 7, 2024

(54) MICROALGAL CULTURE STATE DETERMINATION METHOD

(71) Applicant: HONDA MOTOR CO., LTD., Minato-ku, Tokyo (JP)

(72) Inventors: Mizuho Doi, Wako-shi, Saitama-ken (JP); Nozomi Shiobara, Wako-shi, Saitama-ken (JP); Shohei Kinoshita, Wako-shi, Saitama-ken (JP); Minoru Goto, Wako-shi, Saitama-ken (JP); Kenji Machida, Wako-shi, Saitama-ken (JP); Satoshi Shiozaki, Wako-shi, Saitama-ken (JP)

(21) Appl. No.: 18/685,934

(22) PCT Filed: Jul. 11, 2022
(86) PCT No.: PCT/JP2022/027279

§ 371 Date: Feb. 23, 2024

(30) Foreign Application Priority Data
Sep. 30, 2021 (JP) 2021-160925

(51) Int. Cl. C12Q 1/04; G01N 21/59

(52) U.S. Cl. CPC C12Q 1/04; G01N 21/59

(57) ABSTRACT

In a separation step of a microalgal culture state determination method, a culture liquid, in which a microalga is cultured, is separated into the microalga and a separation liquid. In a transmittance measurement step, the separation liquid is irradiated with light and the transmittance of light to the separation liquid is measured to obtain the transmittance measurement value. In a determination step, the culture state is determined on the basis of the transmittance measurement value.

### TECHNICAL FIELD

The present invention relates to a microalgae culture state assessment method (microalgal culture state determination method).

### BACKGROUND ART

For example, JP 2008-283946 A proposes an assessment method for assessing the proliferative activity of microalgae as the culture state of microalgae. In this assessment method, a culture solution for microalgae is sampled as a test sample. The test sample containing the microalgae is irradiated with light. Thereby, the intensity of fluorescence emitted from the test sample is measured. The intensity of fluorescence obtained by this measurement is checked against data acquired beforehand. This data shows the relationship between the intensity of fluorescence and the proliferative activity. As a result, the proliferative activity of microalgae contained in the culture solution can be quantitatively assessed.

### SUMMARY OF THE INVENTION

In the above assessment method, the sampled test sample contains both living microalgae and dead microalgae (microalgae dead but before decomposition). Until a given time passes after the microalgae die, it is difficult to distinguish between dead and living microalgae. This raises a concern that dead microalgae may emit fluorescence with similar intensity to that of the fluorescence emitted by living microalgae. As a result, there is a concern that the fluorescence emitted by dead microalgae may reduce the accuracy of assessing the proliferative activity of living microalgae. In other words, there is a concern that the above assessment method may be unable to determine the culture state of microalgae with high accuracy.

An object of the present invention is to solve the above-described problems.

One aspect of the present invention is a microalgae culture state assessment method for assessing a culture state of microalgae and includes a separation step of separating a culture solution for culturing the microalgae into the microalgae and a separated solution, a transmittance measurement step of acquiring a transmittance measurement value by irradiating the separated solution with light and by measuring transmittance of the light with respect to the separated solution, and an assessment step of assessing the culture state based on the transmittance measurement value.

In this method for assessing the culture state, the culture state of microalgae can be quantitatively assessed based on the measured transmittance of the separated solution that does not contain microalgae. In this case, for example, it is possible to prevent microalgae in the culture solution from reducing the accuracy of assessing the culture state. Therefore, the culture status of microalgae can be assessed quantitatively and with high accuracy.

### BRIEF DESCRIPTION OF DRAWINGS

FIG. 1 is a step diagram illustrating a microalgae culture state assessment method according to an embodiment of the present invention;

FIG. 2 is a transmission spectrum of the first separated solution for each culture day;

FIG. 3 is a transmission spectrum of the second separated solution for each culture day;

FIG. 4 is a graph showing the relationship between the number of culture days and the light transmittance of the third separated solution for each of microalgae A-D;

FIG. 5 is a graph showing the relationship between the number of culture days and the absorbance of the third separated solution for each of the microalgae A-D;

FIG. 6 is a graph showing the relationship between the number of culture days and the concentration of the whole microalgae in the fourth sampling solution for each of the microalgae A-D;

FIG. 7 is a graph illustrating a transmission spectrum at the start and a transmission spectrum at the end.

### DETAILED DESCRIPTION OF THE INVENTION

The method for assessing a culture state according to the present embodiment assesses the culture state of microalgae cultured in a culture solution. The culture solution is accommodated in, for example, a culture tank (not shown) together with microalgae. The culture solution preferably contains water and nutrients necessary for culturing microalgae. The nutrients are, for example, nitrogen, phosphorus, potassium, etc. The culture tank is placed in an environment capable of receiving light of a wavelength (e.g., 340 to 700 nm) required for the growth of microalgae. One example of such an environment is outdoors where at least one of sunlight and artificial light can be received. Another example of the environment is an indoor environment where at least one of sunlight and artificial light can be received.

The microalgae a culture state of which can be assessed by the method for assessing a culture state are not particularly limited. However, in the case of using the cultured microalgae to produce a biofuel such as ethanol, the microalgae are preferably those that are classified into green algae (for example, Chlamydomonas or Chlorella), prasinophytes, cryptophyceae, and cyanobacteria (for example, Spirulina).

In the method for assessing a culture state according to the present embodiment, as the culture state, the proliferative activity of microalgae is assessed. In the following, an example of applying the method for assessing a culture state according to the present embodiment to select microalgae as seed algae for culturing and producing microalgae will be described. That is, the method for assessing a culture state assesses the proliferative activity of microalgae that will be seed algae. In this case, for example, from experiments conducted beforehand or the like, the proliferative activity at which the microalgae proliferate well and also a predetermined production amount is yielded when the microalgae are cultured as a seed alga is determined beforehand as an assessment reference value.

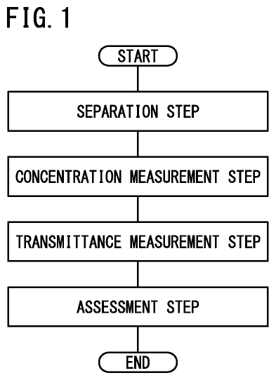

As shown in FIG. 1, the method for assessing a culture state includes a separation step. In this embodiment, microalgae are cultured in the culture solution within the culture tank until a predetermined culture time (e.g., 4 days) appropriate for culturing the seed algae elapses. After the predetermined culture time has elapsed, a portion of the culture solution in the culture tank is sampled to obtain a first sampling solution. In the separation step, the microalgae are separated from the first sampling solution by, for example, centrifugation. Thus, the first sampling solution is separated into microalgae and a separated solution not containing microalgae.

In this embodiment, a concentration measurement step is performed together with the separation step. In the concentration measurement step, part of the culture solution in the culture tank is sampled at the same timing as the timing of obtaining the first sampling solution to obtain the second sampling solution. The concentration of the whole microalgae contained in this second sampling solution is found. The concentration of the whole microalgae contained in the second sampling solution can be treated as the same value as the concentration of the whole microalgae contained in the culture solution in the culture tank after a predetermined culture time has elapsed.

Here, the culture solution in the culture tank (the same as the second sampling solution) contains both living microalgae and dead microalgae before decomposed. Thus, the concentration of the whole microalgae is the concentration in the culture solution of both the living microalgae and the dead microalgae, which is difficult to distinguish from the living microalgae.

As a specific method for obtaining the concentration of the whole microalgae contained in the second sampling solution, the mass of the second sampling solution is first measured. Then, the second sampling solution is filtered, for example, to obtain microalgae separated from the culture solution. The dry mass of this microalgae is measured. Dry mass is the mass of the microalgae in a dried and dehydrated state. The concentration of the whole microalgae in the second sampling solution is obtained from the mass of the second sampling solution measured as described above and the dry mass of the microalgae. That is, the concentration of the whole microalgae contained in the culture solution in the culture tank after a predetermined culture time is obtained.

In the concentration measurement step, the concentration of the whole microalgae contained in the culture solution in the culture tank at the time of starting the culture may be obtained in advance in addition to the concentration of the whole microalgae contained in the culture solution in the culture tank after a predetermined culture time has elapsed. The concentration of the whole microalgae contained in the culture solution in the culture tank at the time of starting the culture can also be obtained in the same manner as the concentration of the whole microalgae contained in the culture solution in the culture tank after a predetermined culture time has elapsed.

After the separation step and the concentration measurement step are completed as described above, the transmittance measurement step is performed. In the transmittance measurement step, by means of, for example, a spectrophotometer, the separated solution is irradiated with light and the light transmittance in the separated solution is measured. This yields transmittance measurement values.

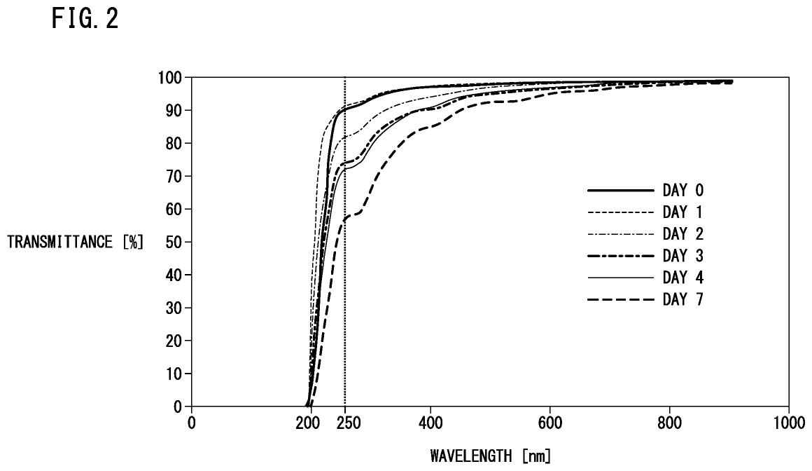

Here, the transmission spectrum of the first separated solution obtained by separating microalgae from the culture solution in which microalgae having a proliferative activity equal to or greater than the assessment reference value are cultured is shown in FIG. 2. The transmission spectrum in FIG. 2 is measured as follows. Microalgae (seed algae) are cultured for 7 days. From the culture start date (day 0) to day 4 of culture, the culture solution in the culture tank is sampled every day to obtain five days' worth of the first separated solution. In addition, the culture solution in the culture tank on day 7 of culture is sampled to obtain the first separated solution. The transmission spectrum of each of these first separated solutions was measured in the wavelength range of 190 to 900 nm.

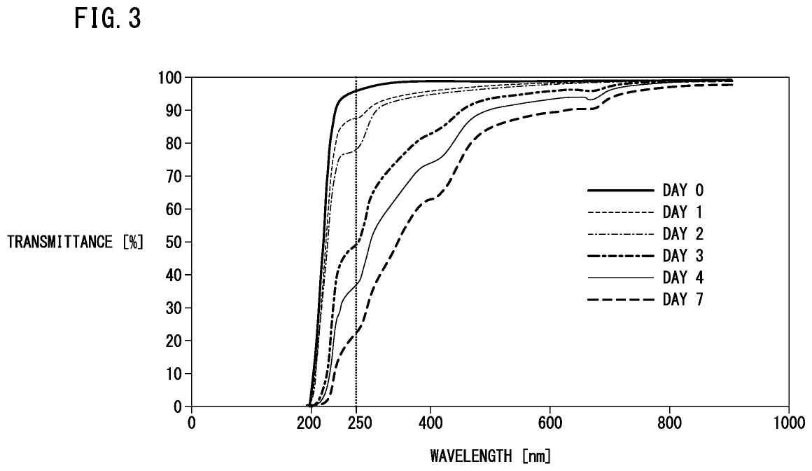

On the other hand, the transmission spectrum of the second separated solution obtained by separating microalgae from the culture solution in which microalgae having a proliferative activity lower than the assessment reference value are cultured is shown in FIG. 3. The transmission spectrum of the second separated solution in FIG. 3 is also measured in the same manner as the transmission spectrum of the first separated solution in FIG. 2.

As shown in FIGS. 2 and 3, the transmittance of both the first and second separated solutions tends to decrease as culture days pass. The amount of decrease in transmittance is larger in the second separated solution than in the first separated solution. That is, the second separated solution obtained by culturing microalgae having the proliferative activity smaller than the assessment reference value exhibits a larger amount of decrease in transmittance with the passage of culture days than the first separated solution obtained by culturing microalgae having the proliferative activity equal to or greater than the assessment reference value. It is considered that this is due to the fact that the amount of decrease in transmittance is proportional to a dead algae concentration. The dead algae concentration is a concentration of dead microalgae.

When the proliferative activity is smaller than the assessment reference value, the ratio of microalgae death to the ratio of microalgae growth is likely to be large. As a result, the dead algae concentration increases due to the higher amount of dead microalgae in the culture solution. It is considered that the transmittance decreases in proportion to the increase in the dead algae concentration. Thus, the amount of decrease in transmittance in the second separated solution is larger than the amount of decrease in transmittance in the first separated solution.

In other words, there is a correlation between the proliferative activity of the microalgae and the transmittance of the separated solution obtained by separating the microalgae from the culture solution in which the microalgae were cultured. Based on this correlation, the lowest value of the transmittance of the separated solution in which the proliferative activity is at the assessment reference value is set as a transmittance reference value, for example, from experiments conducted beforehand. The transmittance reference value is, for example, 70% but is not particularly limited to this value.

In the transmittance measurement step, the wavelength of light with which the separated solution is irradiated is preferably selected from a range of, for example, 230 to 500 nm in order to obtain a transmittance measurement value. It is observed from FIGS. 2 and 3 that when the separated solution is irradiated with light in a wavelength range of 500 nm or less, a decrease in transmittance with the passage of the culture days becomes remarkable. When the separated solution is irradiated with light in a wavelength range of 230 nm or more, a decrease in transmittance with the passage of the culture days can be observed within a range where the influence of errors is weak. Therefore, by selecting from a range of 230 to 500 nm the wavelength of light with which the separated solution is irradiated, it becomes possible to assess the culture state of the microalgae with high accuracy. In the transmittance measurement step of the present embodiment, the separated solution is irradiated with light having a wavelength of 250 nm to obtain a transmittance measurement value.

Next, as shown in FIG. 1, an assessment step is performed. In the assessment step, it is assessed whether or not the proliferative activity of microalgae is equal to or greater than the assessment reference value. Specifically, in the assessment step, the transmittance measurement value obtained in the transmittance measurement step is compared with the transmittance reference value. As described above, by determining the transmittance reference value, it is possible to determine that the proliferative activity of microalgae is equal to or greater than the assessment reference value when the transmittance measurement value is equal to or greater than the transmittance reference value. On the other hand, when the transmittance measurement value is smaller than the transmittance reference value, it is possible to determine that the proliferative activity of microalgae is smaller than the assessment reference value.

In the assessment step, when the proliferative activity of the microalgae is assessed, the concentration of the whole microalgae contained in the culture solution in the culture tank obtained in the concentration measurement step may be further considered. That is, for example, when the concentration of the whole microalgae contained in the culture solution in the culture tank after the passage of a predetermined culture time is equal to or less than the concentration of the whole microalgae contained in the culture solution in the culture tank at the start of the culture, it may be determined that the proliferative activity of the microalgae is smaller than the assessment reference value.

In addition, for example, when the concentration of the whole microalgae contained in the culture solution in the culture tank after the passage of a predetermined culture time is equal to or more than a concentration reference value and the transmittance measurement value is equal to or more than the transmittance reference value, the proliferative activity of microalgae may be determined to be equal to or more than the assessment reference value. Furthermore, for example, when the concentration of the whole microalgae contained in the culture solution in the culture tank after the passage of a predetermined culture time is equal to or greater than the concentration reference value and the transmittance measurement value is smaller than the transmittance reference value, it may be determined that the proliferative activity is smaller than the assessment reference value.

The concentration reference value can be set, for example, from experiments conducted beforehand or the like. The concentration reference value can be, for example, the lowest value of such a concentration of the whole microalgae that allows an assessment that the proliferative activity of the microalgae is equal to or more than the assessment reference value when the microalgae are cultured for a predetermined culture time.

As described above, in the method for assessing a culture state according to the present embodiment, the culture state of the microalgae can be quantitatively assessed based on the transmittance measurement value of the separated solution containing no microalgae. In this case, for example, it is possible to prevent microalgae in the culture solution from reducing the accuracy of assessing the culture state. Therefore, the culture status of microalgae can be assessed quantitatively and with high accuracy.

In the assessment step according to the above embodiment, it is determined, as the assessment of the culture state, whether or not the proliferative activity of microalgae is equal to or greater than the assessment reference value.

In the assessment step of the above embodiment, the transmittance measurement value is compared with the transmittance reference value, and if the transmittance measurement value is equal to or greater than the transmittance reference value, it is determined that the proliferative activity is equal to or greater than the assessment reference value.

However, the method of determining whether the proliferative activity of microalgae is equal to or greater than the assessment reference value based on the transmittance measurement value in the assessment step is not limited to comparing the transmittance measurement value with the transmittance reference value.

For example, in the assessment step, the dead algae concentration may be obtained from the transmittance measurement value. As described above, the amount of decrease in the transmittance of the separated solution with the passage of the culture days is considered to be proportional to the dead algae concentration. Thus, there is a correlation between the transmittance of the separated solution and the dead algae concentration. Therefore, the relationship between the transmittance of the separated solution and the dead algae concentration is obtained as follows, for example, from experiments conducted beforehand or the like.

Four kinds of microalgae with different proliferative activities (microalgae A, B, C, and D) are cultured for 7 days. For each microalgae, from the culture start day (day 0) to day 4 of culture, the culture solution in the culture tank is sampled every day to obtain five days' worth of a third sampling solution. In addition, the culture solution on day 7 of culture is sampled to obtain the third sampling solution. That is, a total of six days' worth of the third sampling solution is obtained for each of the microalgae A-D.

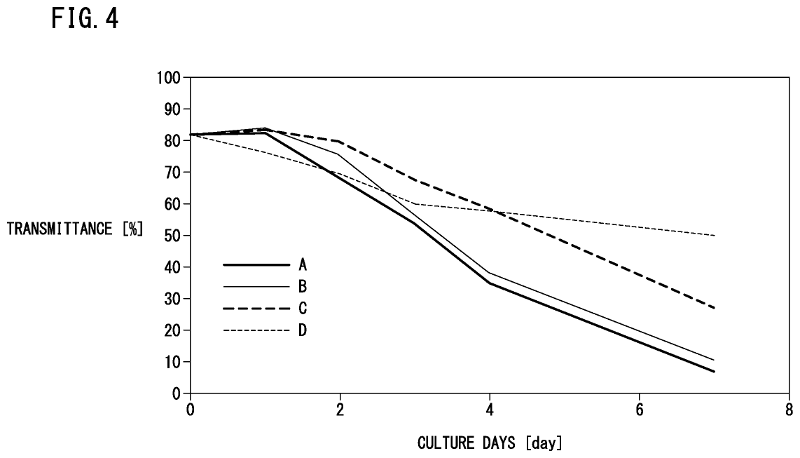

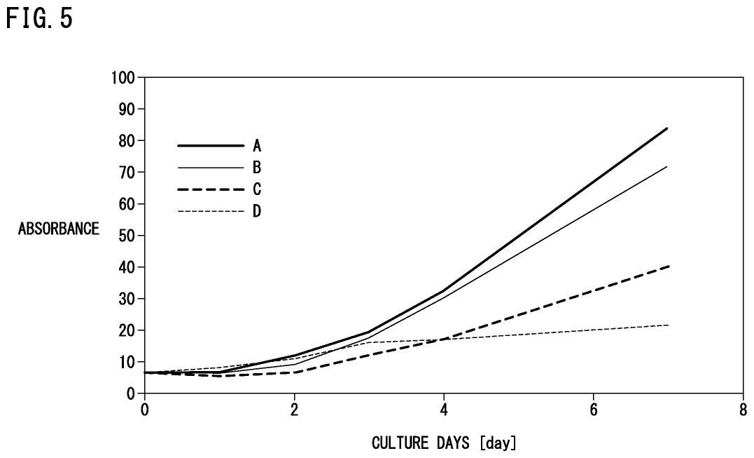

The microalgae contained in each third sampling solution are separated by, for example, centrifugation to obtain a third separated solution which does not contain microalgae. Each of these third separated solution is irradiated with light at a wavelength of 250 nm and the light transmittance of each third separated solution is measured, for example, with a spectrophotometer. FIG. 4 shows the relationship between the number of culture days and the measured light transmittance of each of the third separated solution. The absorbance was also obtained by logarithmic transformation of the light transmittance of each third separated solution. FIG. 5 shows the relationship between the number of culture days and the absorbance of each third separated solution.

At the same timing as the above-mentioned timing of obtaining the third sampling solution, part of the culture solution in the culture tank is sampled to obtain the fourth sampling solution. That is, a total of six days' worth of the fourth sampling solution is obtained for each of the microalgae A-D.

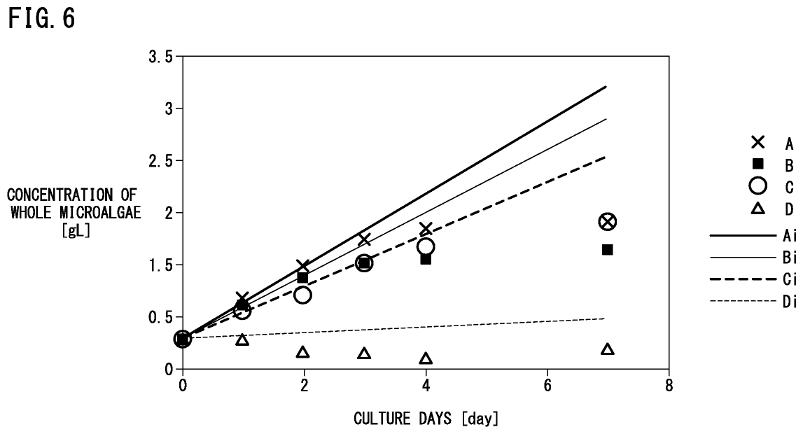

The concentration of the whole microalgae contained in each fourth sampling solution is measured. FIG. 6 shows the relationship between the number of culture days and the measured concentration of the whole microalgae for each of the microalgae A-D. FIG. 6 shows that the rate of increase in concentration of the whole microalgae decreases with increasing culture time. It is considered that in the culture days before the rate of increase decreases, the microalgae are growing ideally. Therefore, the rate of increase in concentration of the whole microalgae (the slope of the graph in FIG. 6) in the number of culture days before the decrease of the rate of increase was defined as an ideal rate of increase.

In FIG. 6, a thick solid line Ai shows an ideal whole microalgae concentration when the microalgae A grows along an ideal rate of increase. A thin solid line Bi shows an ideal whole microalgae concentration when the microalgae B grows along an ideal rate of increase. A thick broken line Ci shows an ideal whole microalgae concentration when the microalgae C grows along the ideal rate of increase. A thin broken line Di shows an ideal whole microalgae concentration when the microalgae D grows along the ideal rate of increase.

A difference between the ideal whole microalgae concentration along the ideal rate of increase and an actually measured whole microalgae concentration is considered to be caused by the death of cultured microalgae. Therefore, the difference between the ideal whole microalgae concentration and the actually measured whole microalgae concentration is defined as the dead algae concentration. That is, as shown in FIG. 6, for example, for microalgae A, the difference between the ideal whole microalgae concentration on the thick solid line Ai and the actually measured whole microalgae concentration (indicated by x in FIG. 6) becomes large after day 4 of culture. The difference between the ideal whole microalgae concentration on this thick solid line Ai and the actually measured whole microalgae concentration is defined as the dead algae concentration of the microalgae A.

From the absorbance of each third separated solution in FIG. 5 and the dead algae concentration in FIG. 6, a conversion coefficient (proportionality constant) for converting the absorbance into the dead algae concentration can be obtained. By obtaining the conversion coefficient in advance in this way, it is possible to obtain the dead algae concentration from the transmittance measurement value acquired in the transmittance measurement step in the assessment step.

In the assessment step, a ratio of the dead algae concentration to the whole microalgae concentration contained in the culture solution in the culture tank obtained in the concentration measurement step is obtained. The ratio of the dead algae concentration to the whole microalgae concentration is compared with a ratio reference value. When the ratio of the dead algae concentration to the whole microalgae concentration is smaller than the ratio reference value, it can be determined that the proliferative activity is equal to or larger than the assessment reference value. On the other hand, when the ratio of the dead algae concentration to the whole microalgae concentration is equal to or larger than the ratio reference value, it can be determined that the proliferative activity is smaller than the assessment reference value.

The ratio reference value can be set, for example, from experiments conducted beforehand. The ratio reference value can be, for example, the lowest value of the ratio of the dead algae concentration to the whole microalgae concentration, at which it can be determined that the proliferative activity of the microalgae is equal to or greater than the assessment reference value when the microalgae are cultured for a predetermined culture time.

That is, in the determining step according to the above exemplary modification, the dead algae concentration, which is the concentration of dead microalgae in the culture solution, is obtained from the transmittance measurement value, and the proliferative activity is determined to be equal to or greater than the assessment reference value when the ratio of the dead algae concentration to the whole microalgae concentration in the culture solution is smaller than the ratio reference value. Even in this case, the culture state of the microalgae can be quantitatively and highly accurately assessed based on the transmittance measurement value of the separated solution that does not contain the microalgae.

As described above, the culture solution in the culture tank contains both living and dead microalgae. This dead microalgae includes those that are difficult to distinguish from the living microalgae. Therefore, there is a concern that the proliferative activity of microalgae cannot be accurately assessed simply from the measurement value obtained by measuring the concentration of the whole microalgae contained in the culture solution. However, in the assessment step according to the exemplary modification of the present embodiment, the dead algae concentration can be obtained from the transmittance measurement value of the separated solution. Therefore, the culture state of the microalgae can be assessed with high accuracy regardless of whether the dead microalgae are contained in the culture solution or not.

In the separation step according to the above embodiment, the separated solution is obtained from the culture solution in which the microalgae are cultured until a predetermined culture time has elapsed, and in the assessment step, the microalgae are determined to be suitable as the seed algae when the proliferative activity is equal to or greater than the assessment reference value. In this case, when it is determined in the assessment step that the proliferative activity is equal to or greater than the assessment reference value, the microalgae cultured until a predetermined culture time has elapsed can be determined to be suitable as the seed algae. Culturing the seed algae thus determined makes it possible to improve a cultured microalgae yield. However, the microalgae whose proliferative activity is determined in the assessment step is not limited to be a seed algae but may be, for example, microalgae during culture production and the like.

The culture state assessed in the assessment step is not limited to the proliferative activity of microalgae. For example, in the assessment step, as the assessment of the culture state, it may be determined whether or not the survival rate of the microalgae in the culture solution is equal to or greater than an assessment reference value.

That is, in the assessment step, the dead algae concentration, which is the concentration of dead microalgae in the culture solution, is obtained from the transmittance measurement value; the survival rate, which is the ratio of the surviving microalgae to the whole microalgae in the culture solution, is obtained from the concentration of the whole microalgae in the culture solution and the dead algae concentration; and it may be determined whether or not the survival rate is equal to or greater than the assessment reference value as the assessment of the culture state.

From the difference between the dead algae concentration and the whole microalgae concentration in the culture solution, it is possible to determine the concentration of living microalgae. Thus, it is possible to determine the survival rate of microalgae in the culture solution. For example, when the survival rate of microalgae is equal to or greater than the judgment reference value, it can be judged that the microalgae are well cultured because the culture solution contains sufficient living microalgae. On the other hand, for example, when the survival rate of microalgae is smaller than the judgment reference value, it can be judged that there is a concern that the culture state of microalgae may be problematic because dead microalgae in the culture solution are increasing.

In the microalgae culture state assessment method according to the above embodiment, the wavelength of the light emitted to the separated solution in the transmittance measurement step is preferably 230 nm or more. In this case, as shown in FIGS. 2 and 3, a decrease in transmittance with the passage of the culture days can be observed within a range in which the influence of errors is small. As a result, it becomes possible to assess the microalgae culture state with high accuracy.

In the microalgae culture state assessment method according to the above embodiment, the wavelength of the light emitted to the separated solution in the transmittance measurement step is preferably 500 nm or less. In this case, as shown in FIGS. 2 and 3, a decrease in transmittance with the passage of the culture days can be observed in a range where the decrease in transmittance is significantly observed. As a result, it becomes possible to assess the microalgae culture state with high accuracy.

The wavelength of the light emitted onto the separated solution in the transmittance measurement step may be determined by performing a wavelength determination step before the transmittance measurement step. In this case, the microalgae culture state assessment method for assessing the culture state of microalgae includes, before the transmittance measurement step, a wavelength determination step of culturing microalgae for detection in a culture solution for detection, and determining the wavelength of light with which a separated solution is irradiated in the transmittance measurement step, wherein the wavelength determination step includes a first separation step of separating the culture solution for detection given when the culture of microalgae for detection is started into the microalgae for detection and a separated solution at start, a second separation step of separating the culture solution for detection given when the culture of microalgae for detection in the culture solution for detection is ended into the microalgae for detection and a separated solution at end, a first acquisition step of acquiring a transmission spectrum at start from a transmittance obtained by irradiating the separated solution at start with light in a predetermined range of wavelength, a second acquisition step of acquiring a transmission spectrum at end from the transmittance obtained by irradiating the separated solution at end with light in a predetermined range of wavelength, and a selection step of selecting the wavelength of light with which the separated solution is irradiated in the transmittance measurement step, wherein the selection step includes a step of dividing the transmission spectrum at start into two regions including a first region in which the transmittance increases sharply against the increase in wavelength and a second region in which the transmittance increases slowly against the increase in wavelength compared with the first region, and identifying a first wavelength at the boundary point between the first region and the second region, and a step of dividing the transmission spectrum at end into two regions including a third region in which the transmittance increases sharply against the increase in wavelength and a fourth region in which the transmittance increases slowly against the increase in wavelength compared with the third region, and identifying a second wavelength at a boundary point between the third region and the fourth region, and a step of selecting the wavelength of light with which the separated solution is irradiated from a wavelength region between the first wavelength and the second wavelength.

Specifically, a wavelength determination step is performed in advance before the transmittance measurement step. In the wavelength determination step, the microalgae for detection are cultured in the culture solution for detection for a predetermined number of days (e.g., 7 days). In the first separation step, the culture solution at the start of the culture is sampled and separated into the microalgae for detection and the separated solution at the start. In the second separation step, the culture solution at the end of the culture is sampled and separated into the microalgae for detection and the separated solution at the end.

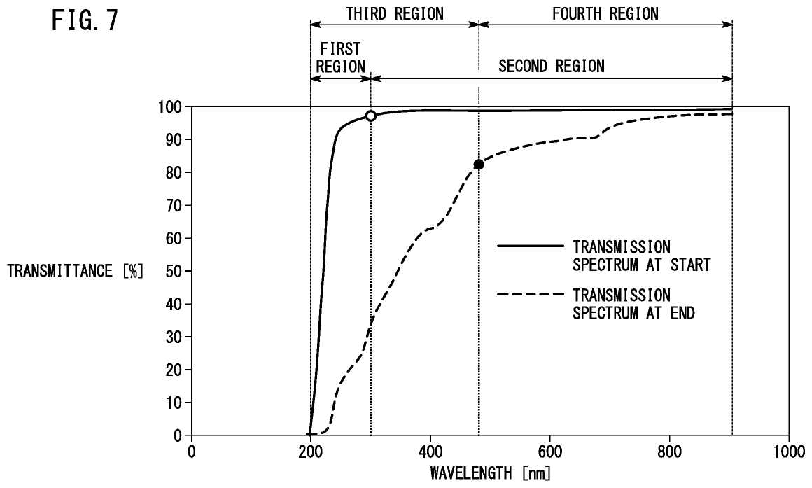

In the first acquisition step, a transmission spectrum at the start (FIG. 7) is acquired from a transmittance obtained by irradiating the separated solution at the start with light in a predetermined range of wavelength (e.g., 190 to 900 nm). In the second acquisition step, the transmission spectrum at the end (FIG. 7) is acquired from the transmittance obtained by irradiating the separated solution at the end with light in a predetermined range of wavelength (e.g., 190 to 900 nm).

In the selection step, the wavelength of light with which the separated solution is irradiated in the transmittance measurement step is selected using the transmission spectrum at the start and the transmission spectrum at the end acquired as described above. Specifically, in the selection step, the transmission spectrum at the start is divided into two parts, i.e., a first region and a second region, as shown in FIG. 7. The first region exhibits a sharp increase in transmittance against the increase in wavelength compared to the second region. The second region has a gradual increase in transmittance against the increase in wavelength compared to the first region. The wavelength at the boundary point between the first region and the second region, which is indicated by a white circle in FIG. 7, is specified as the first wavelength.

In the selection step, the transmission spectrum at the end is divided into a third region and a fourth region. The third region shows a sharp increase in transmittance against the increase in wavelength compared to the fourth region. The fourth region has a gradual increase in transmittance against the increase in wavelength compared to the third region. The wavelength at the boundary point between the third region and the fourth region, which is indicated by a black circle in FIG. 7, is specified as the second wavelength.

The wavelength of the light with which the separated solution is irradiated in the transmittance measurement step is selected from a wavelength region between the first and second wavelengths specified as described above. In this way, by selecting the wavelength of light with which the separated solution is irradiated in the transmittance measurement step, a decrease in transmittance with the passage of the culture days can be observed within a range that is less affected by errors. In addition, the decrease in transmittance with the passage of the culture days can be observed in a range where the decrease in transmittance is significantly observed. As a result, it becomes possible to assess the microalgae culture state with high accuracy.

The present invention is not limited to the above disclosure, and various configurations can be employed without departing from the gist of the present invention.

What is claim is:

1. A microalgae culture state assessment method for assessing a culture state of microalgae, comprising:
a separation step of separating a culture solution for culturing the microalgae into the microalgae and a separated solution;
a transmittance measurement step of acquiring a transmittance measurement value by irradiating the separated solution with light and by measuring transmittance of the light with respect to the separated solution; and
an assessment step of assessing the culture state based on the transmittance measurement value.

2. The microalgae culture state assessment method according to claim 1, wherein
in the assessment step, it is determined whether proliferative activity of the microalgae is equal to or greater than an assessment reference value as an assessment of the culture state.

3. The microalgae culture state assessment method according to claim 2, wherein
in the assessment step, the transmittance measurement value is compared with a transmittance reference value, and it is determined that the proliferative activity is equal to or greater than the assessment reference value when the transmittance measurement value is equal to or greater than the transmittance reference value.

4. The microalgae culture state assessment method according to claim 2, wherein
in the assessment step, a dead algae concentration, which is a concentration of dead microalgae in the culture solution, is obtained from the transmittance measurement value, and it is determined that the proliferative activity is equal to or greater than the assessment reference value when a ratio of the dead algae concentration to a concentration of a whole microalgae in the culture solution is smaller than a ratio reference value.

5. The microalgae culture state assessment method according to claim 2, wherein
in the separation step, the separated solution is obtained from the culture solution in which the microalgae are cultured until a predetermined culture time elapses, and
in the assessment step, it is assessed that the microalgae are suitable as seed algae when the proliferative activity is equal to or greater than the assessment reference value.

6. The microalgae culture state assessment method according to claim 1, wherein
in the assessment step, a dead algae concentration, which is a concentration of dead microalgae in the culture solution, is obtained from the transmittance measurement value; a survival rate, which is a ratio of surviving microalgae to a whole microalgae in the culture solution, is obtained from a concentration of the whole microalgae in the culture solution and the dead algae concentration; and it is determined whether the survival rate is equal to or greater than an assessment reference value as an assessment of the culture state.

7. The microalgae culture state assessment method according to claim 1, wherein
a wavelength of the light with which the separated solution is irradiated in the transmittance measurement step is 230 nm or more.

8. The microalgae culture state assessment method according to claim 1, wherein
a wavelength of the light with which the separated solution is irradiated in the transmittance measurement step is 500 nm or less.

9. The microalgae culture state assessment method according to claim 1, further comprising,
before the transmittance measurement step, a wavelength determination step of culturing microalgae for detection in a culture solution for detection and determining a wavelength of the light with which the separated solution is irradiated in the transmittance measurement step,
wherein
the wavelength determination step includes:
a first separation step of separating the culture solution for detection given when the culturing of the microalgae for detection in the culture solution for detection is started into the microalgae for detection and a separated solution at start;
a second separation step of separating the culture solution for detection given when the culture of the microalgae for detection in the culture solution for detection is ended into the microalgae for detection and a separated solution at end;
a first acquisition step of acquiring a transmission spectrum at start from a transmittance obtained by irradiating the separated solution at start with light in a predetermined range of wavelength;
a second acquisition step of acquiring a transmission spectrum at end from a transmittance obtained by irradiating the separated solution at end with light in a predetermined range of wavelength; and
a selection step of selecting a wavelength of the light with which the separated solution is irradiated in the transmittance measurement step, and
the selection step includes:
a step of dividing the transmission spectrum at start into two regions including a first region in which the transmittance increases sharply with an increase in wavelength and a second region in which the transmittance increases slowly with an increase in wavelength compared with the first region, and identifying a first wavelength at a boundary point between the first region and the second region;
a step of dividing the transmission spectrum at end into two regions including a third region in which the transmittance increases sharply with an increase in wavelength and a fourth region in which the transmittance increases slowly against an increase in wavelength compared with the third region, and identifying a second wavelength at a boundary point between the third region and the fourth region; and
a step of selecting the wavelength of the light with which the separated solution is irradiated from a wavelength region between the first wavelength and the second wavelength.
